(19) United States
(12) Patent Application Publication
(10) Pub. No.: US 2015/0272892 A1
(43) Pub. Date: Oct. 1, 2015

(54) DISSOVLABE TABLET

(71) Applicant: Jeffery K. SLABODEN, (US)

(72) Inventor: Jeffery K. Slaboden, Watsonville, CA (US)

(21) Appl. No.: 14/428,416

(22) PCT Filed: Sep. 13, 2013
(86) PCT No.: PCT/US2013/059784

§ 371 Date: Mar. 16, 2015

Related U.S. Application Data
(63) Continuation-in-part of application No. 14/026,795, filed on Sep. 13, 2013.
Application No. 14/026,795 is a continuation-in-part of application No. 13/621,109, filed on Sep. 15, 2012, now abandoned.

(51) Int. Cl. A61K 9/20

(52) U.S. Cl. CPC A61K 9/2072

(57) ABSTRACT

A dissolvable tablet is disclosed. The tablet includes a front side, an obverse side and a thickness between the front side and the obverse side. The front side and obverse side have a variation of convex and concave surfaces. The convex and concave surfaces allow for a plurality of dissolvable tablets to be stacked for packaging. Further, the variation of convex and concave surfaces allow for a self-agitation when the dissolvable tablet is dissolved within a liquid.

### REFERENCE TO PENDING APPLICATIONS

This application claims the benefit of International Application No. PCT/US13/59784 filed on Sep. 13, 2013, U.S. patent application Ser. No. 13/621,109 filed on Sep. 15, 2012 and U.S. patent application Ser. No. 14/026,795 filed on Sep. 13, 2013.

### REFERENCE TO MICROFICHE APPENDIX

This application is not referenced in any microfiche appendix.

### BACKGROUND OF THE INVENTION

The present invention is generally directed toward a dissolvable tablet, and more specifically, toward a dissolvable tablet having an improved disintegration time. The improved disintegration time is provided by maximum surface contact with liquid and in one variant by adding an effervescent additive to the tablet combined with the design creates an increased agitation action allowing the tablet to dissolve and mix more readily.

Many tablets are effervescent tablets which are designed to break apart in contact with water or another liquid, releasing carbon dioxide in the process. The application of these tablets can be used to deliver drugs and enzymes, flavor drinks, add fragrance, as well as being used with dish detergent and laundry products. No existing tab harnesses/captures the resulting CO2 gas to create motion in the tablet and flip it over and over or allow a clam shell type action to help in the dissolving of the tablet.

These tablets are produced by the compression of component ingredients into a dense mass, which is packaged in an container, jar, pouch or a blister pack. When needed, the tablets can be dropped into water or another liquid to make a solution. For example, cleaning tablets may be added to laundry or filled tubs of water, depending on the package directions.

From a drug delivery perspective, there are several advantages to effervescent tablets. One of the biggest advantages is that they deliver drugs to the body rapidly, because the drug is delivered in the form of a solution that is easy to absorb. Dosage control also is easier, and effervescent tablets can be used to protect certain ingredients from the highly acidic environment of the stomach, so that the body has a chance to access them.

These types of tablets are made from an active ingredient, binder material and a combination of an acid and base to create the carbon dioxide reaction when placed in contact with water. Typical acids can include citric, malic, tartaric, adipic, and fumaric. Typical bases used in the effervescent reaction are sodium bicarbonate, potassium bicarbonate, sodium carbonate, sodium sesquacarbonate and potassium carbonate. The term active ingredient as used herein means the substance that is active. For example, in a pharmaceutical drug tablet, the active ingredient that substance that is biologically active.

Binders are normally necessary in effervescent tablets to bring the tablet hardness to a point where handling is possible. These binders should be water-soluble and include dextrose, sorbitol, xyitol, lactose and/or cellulose derivatives. The ideal amount of binder is one that makes the tablet hard enough to handle, but soft enough to disintegrate quickly (the harder the tablet, the slower the disintegration) and dry enough to be stable.

There are several categories of effervescent tablets' active ingredients, that include: those that are difficult to digest or disruptive to the stomach; those that are pH-sensitive, such as amino acids and antibiotics; those requiring a large dose; and those that are susceptible to light, oxygen, or moisture.

Traditional effervescent tablets are produced in much the same manner as conventional tablets through the use of a tablet press that can deliver high compression forces. A tablet press is a mechanical device that compresses powder into tablets of uniform size and weight. To form a tablet, the granulated material must be metered into a cavity formed by two punches and a die, and then the punches must be pressed together with great force to fuse the material together into a dense mass.

One of the drawbacks of the prior art of dense flat tablets is that the disintegration time of this tablet after it has been placed in a container of water or other fluid. The traditional flat disk shape of tablets and related density contributes to this delay. Once the tablet is introduced into the fluid, it settles to the bottom of the container. On the bottom of the container, only one side of the tablet is fully exposed to the fluid increasing the time for the tablet to disintegrate. No existing tablet harnesses or captures the resulting carbon dioxide gas to create a agitation motion in the tablet allowing it to flip it over and over or allow a clam shell type action to help in the dissolving of the tablet. Therefore, there is a need for a tablet that addresses the shortcomings of the prior art.

### SUMMARY OF THE INVENTION

The inventive dissolvable tablet described herein satisfies the needs set out above.

In one aspect of the present invention, a dissolvable tablet is disclosed. The tablet includes a front/top side having a concave surface and an obverse/bottom side having a convex surface. The tablet also has a thickness. The shape of the tablet can take the traditional round shape or can include other geometrical shapes, such as, oval, rectangular, triangular, non-traditional and custom shapes. Other aspects of the present invention include both the dissolvable tablet's front side and obverse side having variations of concave and convex surfaces.

Upon reading the included description, various alternative embodiments will become obvious to those skilled in the art. These embodiments are to be considered within the scope and spirit of the subject invention, which is only limited by the claims which follow and their equivalents.

### BRIEF DESCRIPTION OF THE DRAWINGS

FIG. 1 is a perspective view of an embodiment of the present invention.

FIG. 2 is a top view of the embodiment of the present invention shown in FIG. 1.

FIG. 3 is a cross-sectional side view of the embodiment of the present invention along line AA as shown in FIG. 2.

FIG. 4 is a perspective view of a plurality of the embodiment of the present invention shown in FIG. 1 is a stacked configuration.

FIG. 5 is a side view of the plurality of the embodiment of the present invention shown in FIG. 4.

FIG. 6 is a cross-sectional side view of the plurality of the embodiment of the present invention along line AA as shown in FIG. 4.

FIG. 7 is a perspective view of an additional embodiment of the present invention.

FIG. 8 is a side view of the embodiment of the present invention shown in FIG. 7 along direction A.

FIG. 9 is a side view of the embodiment of the present invention shown in FIG. 7 along direction B.

FIG. 10 is a cross-sectional side view of the embodiment of the present invention shown in FIG. 7.

FIG. 11 is a top view of the embodiment of the present invention shown in FIG. 7.

FIG. 12 is a perspective view of a plurality of the embodiment of the present invention shown in FIG. 7 is a stacked configuration.

### DESCRIPTION OF THE INVENTION

The present invention is generally directed toward a dissolvable tablet, and more specifically, toward a dissolvable tablet having an improved disintegration time.

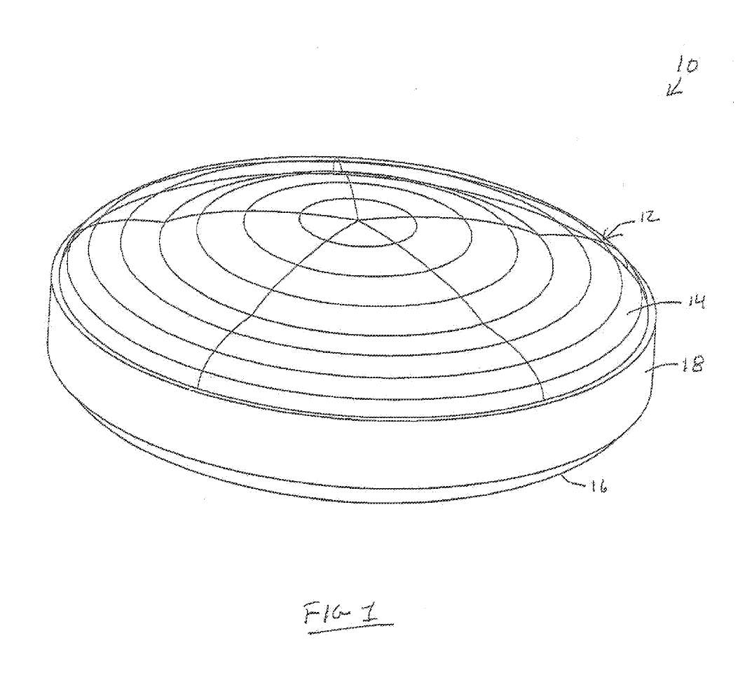

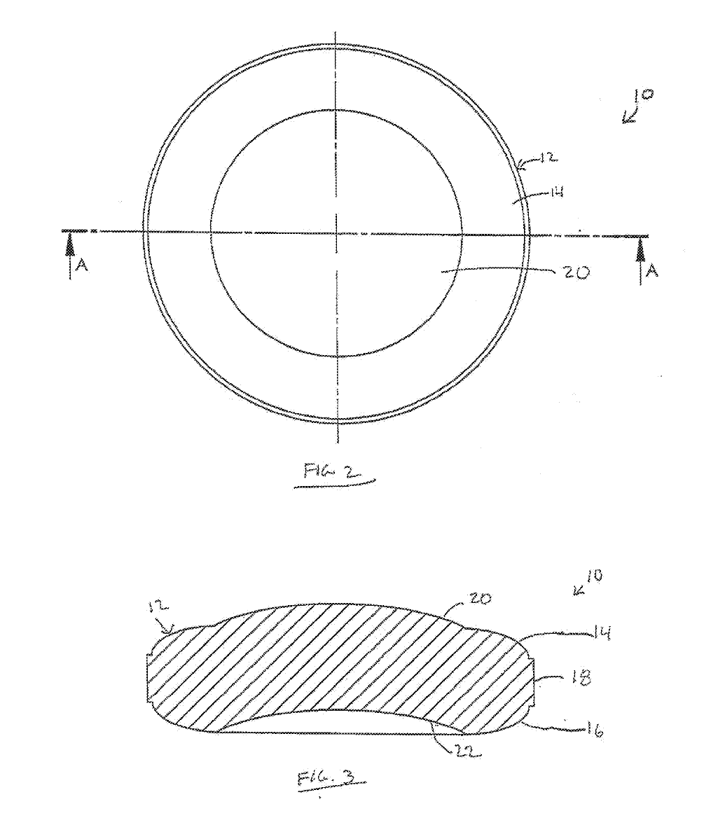

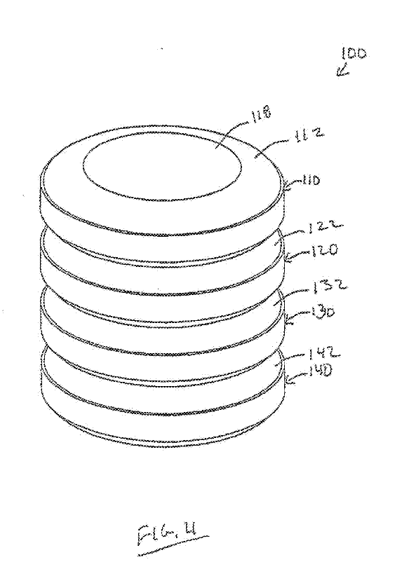

As illustrated in FIGS. 1-3, an embodiment 10 of the present invention includes a dissolvable tablet 12 having a front side 14, an obverse side 16 and a thickness 18. Front side 14 can also be referred to as the top side while obverse side 16 can be referred as the bottom side.

Front side 14 has a convex surface portion 20 that curves outward. When tablet 12 is placed in a fluid-filled container (not shown), such as water, the amount of time needed to dissolve is dependent upon the amount of surface area of tablet 12 that is exposed to the fluid. By having convex surface portion 20, if tablet 12 lands front side down, the entire front side 14 will not be in contact with bottom of the container. Convex surface portion 20 will ensure that only a small portion of front side 14 is in contact with the bottom of the container while providing a greater amount of surface area of tablet 12 is exposed to the fluid. This allows for a faster disintegration time for tablet 12.

Obverse side 16 has a concave surface portion 22 curves inward. In a manner similar to convex surface portion 20, concave surface portion 22 allows for the surface area of obverse side 16 to be exposed to water in the event tablet 12 lands obverse side down.

The shape of embodiment 10 is round, however, those skilled in the art will recognize this is illustrative and not meant to be limiting. The shape can take other geometric shapes, including but not limited to such as, oval, rectangular, triangular, non-traditional and custom shapes.

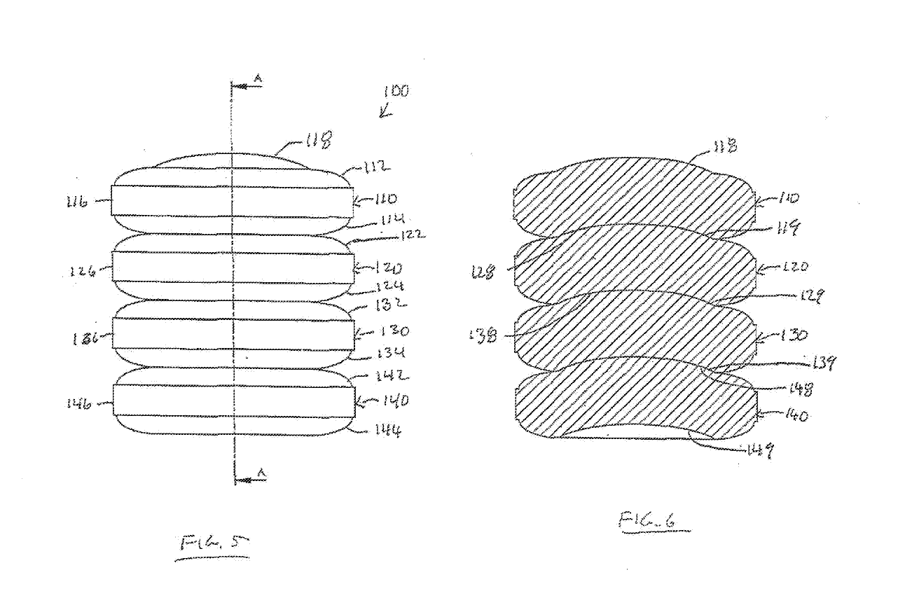

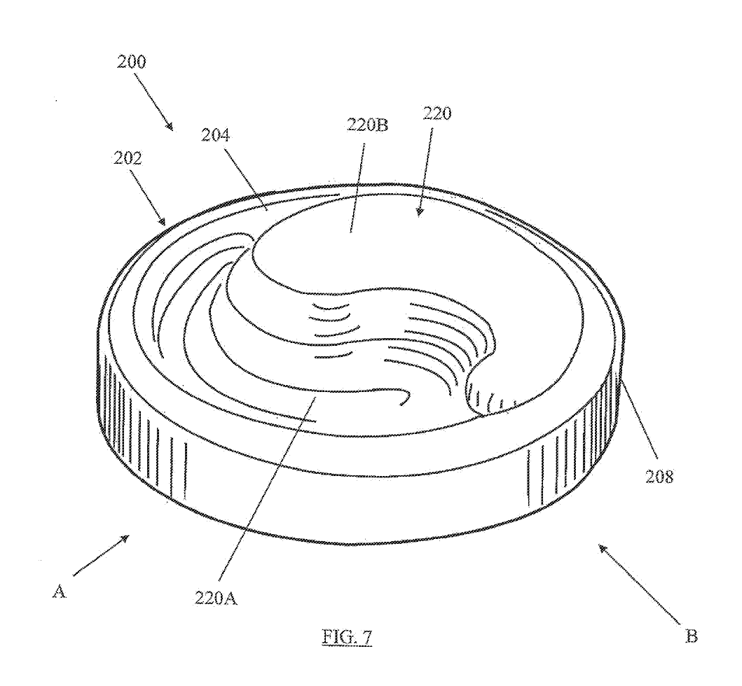

Another benefit of the inventive tablet allows for an improvement of their transportation. As illustrated in FIG. 4-6, a stack of dissolvable tablets 100 is shown. This stack 100 includes four individual tablets 110, 120, 130 and 140. Each tablet has a front side, 112, 122, 132 and 142, with a concave surface, 118, 128, 138 and 148, and an obverse side, 114, 124, 134 and 144 with a convex surface, 119, 129, 139 and 149. As show in the Figures, the obverse sides of the tablets nest in the front sides of the tablets, securing them. This feature allows for the stacking of many tablets without the stack sliding apart. Further, the stack of tablets 100 can be packaged in a tube or other similar container.

The use of four dissolvable tablets is for illustration and those skilled in the art will recognize that stacks of the dissolvable tablet can include less than or more than four tablets.

Another embodiment of the present invention relates to the manufacturing of the inventive tablet and related method. The inventive tablet is formed through traditional manufacturing methods, such as by the use of a tablet press. This tablet press creates the tablet through the combined pressing action of two punches and a die. In the first step of a typical operation, the bottom punch is lowered in the die creating a cavity into which the granulated feedstock is fed. The exact depth of the lower punch can be precisely controlled to meter the amount of powder that fills the cavity. The excess is scraped from the top of the die, and the lower punch is drawn down and temporarily covered to prevent spillage. Then, the upper punch is brought down into contact with the powder as the cover is removed. The force of compression is delivered by high pressure compression rolls or hydraulic pressure which fuse the granulated material together into a hard tablet. After compression, the lower punch is raised to eject the tablet.

Each of the upper and lower punches have an flat head, working length and punch cup. It is the curvature of each of the punch cups gives the tablet its shape. In the present invention, the punch cups are modified to create the convex surface portion 20 and concave surface portion 22 on the inventive tablet.

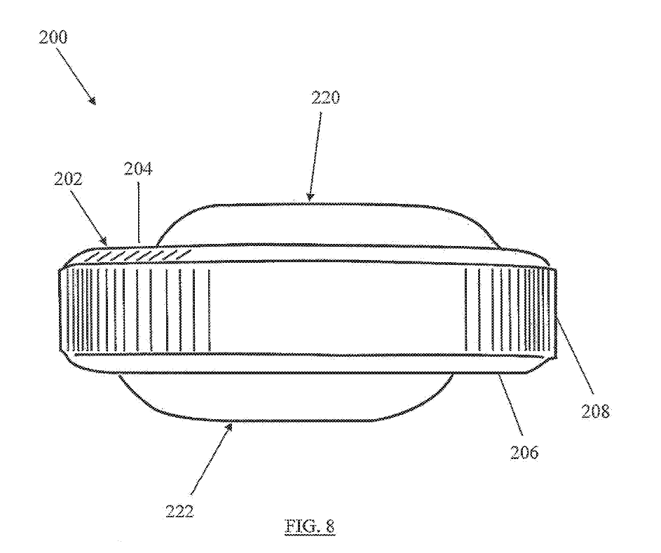

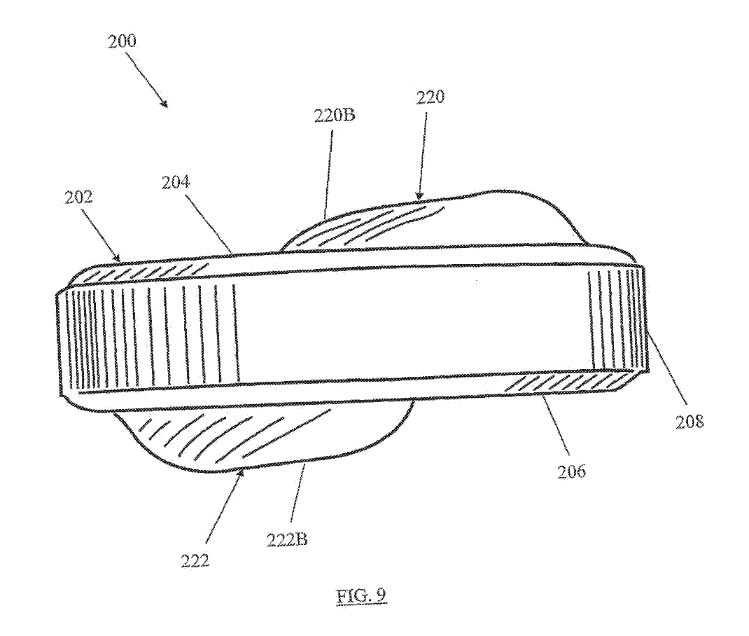

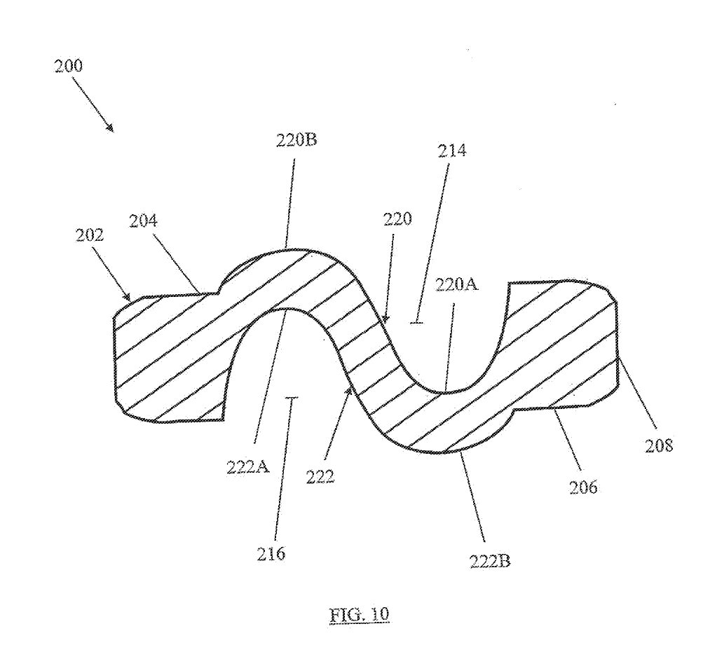

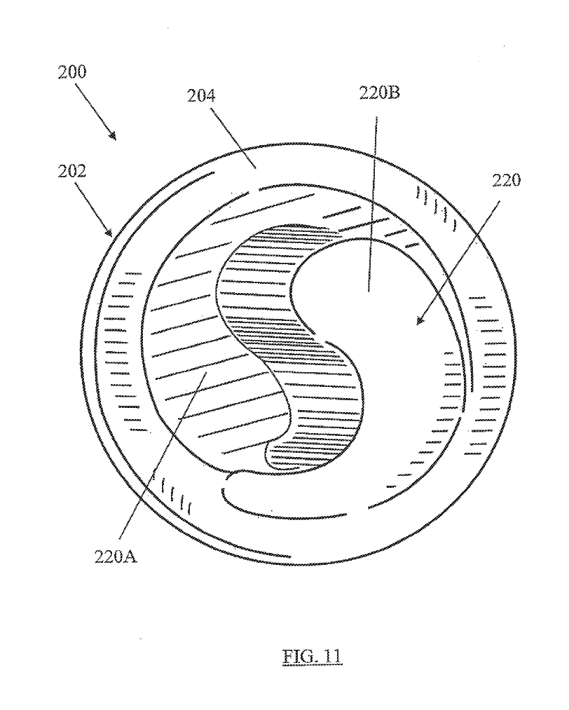

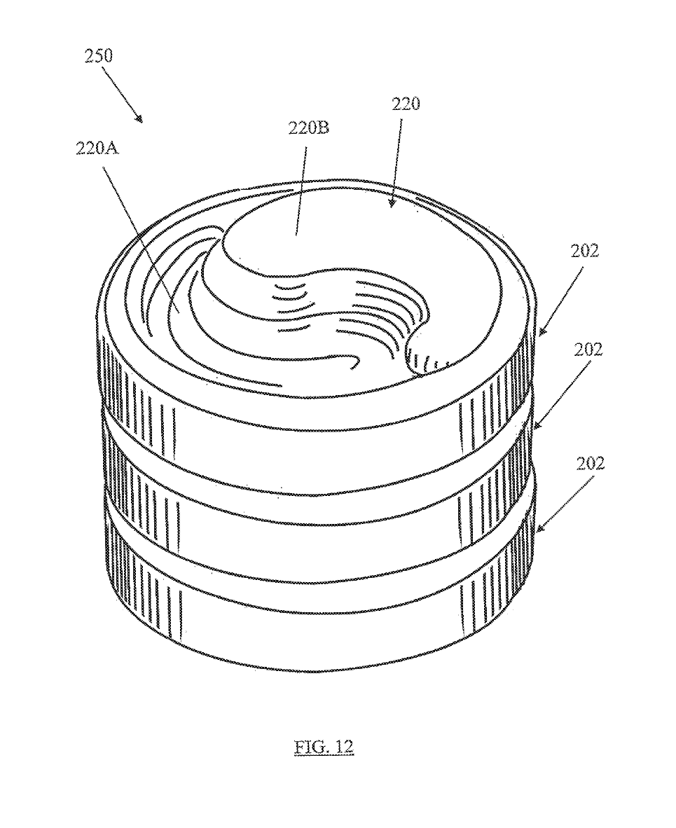

As illustrated in FIGS. 7-11, an embodiment 200 of the present invention includes a dissolvable tablet 202 having a front side 204, an obverse side 206 and a thickness 208. Front side 204 can also be referred to as the top side while obverse side 206 can be referred as the bottom side.

Both front side 204 and obverse side 206 has a combination concave/concave surface 220, 222 that curves outward 220A, 222A and inward 220B, 222B creating an edge 210, 212 around the circumference of tablet 202. When tablet 202 is placed in a fluid-filled container, not shown, tablet 202 will come to rest on the bottom of the container. In so doing, either edge 210 or 212 will come in contact with the bottom forming a cavity 214, 216 between tablet 202 and the bottom of the container. When tablet 202 dissolves, carbon dioxide is released into cavity 214, 216 increasing the pressure therein. After a sufficient amount of carbon dioxide is captured within cavity 214, 216, tablet 202 is lifted off of the bottom of the container releasing the captured carbon dioxide. This often results in the tablet lifting completely off the bottom of the container and flipping over exposing the opposite side when settling to the bottom Tablet 202 then settles once again on the bottom of the container and thereby starting the carbon dioxide capture/release process over again.

The repeated rising and falling of tablet 202 within the container acts to agitate the fluid. This agitation acts to more efficiently and quickly mix and combine the active ingredient within tablet 202 with the fluid within container.

Depending of the ratio between the size of cavity 214, 216 and the weight of tablet 202, the motion of tablet 202 can be controlled. When tablet 202 is of a greater weight compared to the size of cavity 214, 216, the carbon dioxide gas that trapped therein would not be sufficient to cause tablet 202 to rise off the bottom of the container. Likewise, when tablet 202 is of a lighter weight compared to the size of cavity 214, 216, the carbon dioxide gas that trapped therein would be sufficient to cause tablet 202 to rise off the bottom of the container. As the weight of tablet 202 is dependent upon its density and its size, the denser and/or larger the size of tablet 202 is compared to the size of cavity 214, 216 will affect the ability of tablet 202 to rise off of bottom of the container.

As illustrated in FIG. 12, a stack of dissolvable tablets 250 made from a plurality of tablets 202 is shown. The stack of tablets 250 are stacked so that the outward curve 220A of a first one of the plurality of tablets 202 is nested with the inward curve 220B of a second one of the plurality of the tablets 202. This allows all the individual tablets 202 be nested and thereby secured to each other. This feature allows for the stacking of many tablets without the stack sliding apart. The stack of tablets can then be packaged in a tube or other similar container.

In the Figures, the shape of tablet 202 is illustrated as being round, however, those skilled in the art will recognize this is illustrative and not meant to be limiting. The shape can take other geometric shapes, including but not limited to such as, oval, rectangular, triangular, non-traditional and custom shapes. Further, those skilled in the art will recognize that the use of four tablets is merely illustrative and not meant to be limiting.

While embodiments of the present invention have been illustrated and described, such disclosures should not be regarded as any limitation of the scope of our invention. The true scope of our invention is defined in the appended claims. Therefore, it is intended that the appended claims shall be construed to include both the preferred embodiment and all such variations and modifications as fall within the spirit and scope of the invention.

We claim:

1. A dissolvable tablet comprising:
a tablet body having a front side, an obverse side and a thickness there between,
the front side having a convex surface, and
the obverse side having a convex surface.

2. The dissolvable tablet of claim 1, wherein the tablet body comprises:
an active ingredient, binder material and a combination of an acid and base to create a chemical reaction that creates carbon dioxide when placed in contact with liquid.

3. The dissolvable tablet of claim 1, wherein the tablet body comprises:
the front side having a combination concave/convex surface, and
the obverse side having a combination concave/convex surface.

4. The dissolvable tablet of claim 1, wherein the tablet body comprises:
the front side having a concave surface, and
the obverse side having a concave surface.

5. A stack of dissolvable tablets comprising:
a plurality of a first tablets with each tablet having a first front side, a first obverse side and a first thickness there between, each of the first front sides and first obverse sides having a convex surface; and
a plurality of a second tablets with each tablet having a second front side, a second obverse side and a second thickness there between, each of the second front sides and second obverse sides having a concave surface,
wherein the plurality of first tablets is alternatively arranged between the plurality of second tablets so that the concave surfaces of each of the second tablets nest with the convex surfaces of the first tablets.

6. The dissolvable tablet of claim 5, wherein the tablet body comprises:
an active ingredient, binder material and a combination of an acid and base to create a carbon dioxide reaction when placed in contact with water.

7. A stack of dissolvable tablets comprising:
a plurality of a tablets with each tablet having a front side, a obverse side and a thickness there between, each of the front sides and obverse sides having a combination concave/convex surface,
wherein each of the plurality of tablets is arranged so that the concave portion of each surface nests with the convex portion of each surface of the adjacent tablet.

8. The dissolvable tablet of claim 7, wherein the tablet body comprises:
an active ingredient, binder material and a combination of an acid and base to create a carbon dioxide reaction when placed in contact with liquid.
